(19) United States
(12) Patent Application Publication
(10) Pub. No.: US 2017/0055874 A1
(43) Pub. Date: Mar. 2, 2017

(54) DEVICE, SYSTEM AND METHOD FOR INTRAVESICAL URODYNAMIC ANALYSIS

(71) Applicants: Eduard PAPIROV, Hod-HaSharon (IL); Shiri MIZRACHI, Rananna (IL)

(72) Inventors: Eduard Papirov, Hod-HaSharon (IL); Shiri Mizrachi, Ra'anana (IL)

(21) Appl. No.: 15/120,119

(22) PCT Filed: Feb. 19, 2015
(86) PCT No.: PCT/IB2015/051282

§ 371 Date: Aug. 18, 2016

(30) Foreign Application Priority Data
Feb. 19, 2014 (GB) 1402958.1

(51) Int. Cl. A61B 5/07; A61B 8/08; A61B 5/0488; A61B 5/20

(52) U.S. Cl. CPC A61B 5/07; A61B 5/204; A61B 8/08; A61B 5/04882

(57) ABSTRACT

The present invention relates to a device, system and a method for urodynamic analysis in non-clinical setting and/or natural settings allowing the urinary system to function under normal physiological conditions. The device configured for placement within the urinary bladder for performing intravesical urodynamic measurements, the device comprising an external housing provided from biocompatible materials; a sensor module including a plurality of ultrasound sensors, and at least one pressure transducer, the sensor module functionally coupled with electronic circuitry including a communication module; memory module, and a controller module.

### FIELD OF THE INVENTION

The present invention relates to a device, system and a method for urodynamic analysis, and in particular, to such a device, system and method that provides urodynamic analysis in non-clinical setting and/or natural settings allowing the urinary system to function under normal physiological conditions.

### BACKGROUND OF THE INVENTION

The urinary bladder functions to collects and stores urine produced by the kidneys. The urinary bladder received urine from the kidney via the upper ureters, where it is stored until it is urination. The urinary bladder excretes the urine via the urethra. Excretion of urine from the urinary bladder is a controlled function based on the concerted activity of the bladder muscles and the urinary sphincters. Control over the muscles along the urinary path from the bladder to the urethra is termed continence while lack of control of the urinary pathway is referred to as incontinence. For purposes herein the inability of the body to control the discharge of urine is termed incontinence.

Incontinence, may be due to various reasons with a variety of origins usually either relating to at least one or a combination of neurological origins and/or physiologic origins. An example of physiologic original is seen in women in the form of poor muscle tone of the pelvic floor, leading to incontinence. In men, incontinence problems are usually associated with the prostate gland where urinary retention issues are usually due to or associated with the prostate gland itself.

In diagnosing incontinence a variety of urodynamic testing is performed in attempting to ascertain the urinary disorder at hand in an attempt to reach. Urodynamic studies includes various tests, studies and observations of bladder pressure measurement, abdominal pressure measurements, urine flows, electromyography (EMG) signals of the muscles of urogenital area, medical imagery such as X-rays, ultrasounds; uro-flow analysis.

Urodynamic testing produces graphical and numerical data that record the test data to provide that may be further analyzed allowing a practitioner to diagnose and attempt to identify and categorized the problem while attempting to define a potential remedy. Urodynamic testing is generally provided in a clinical setting which at times may be problematic for some individuals.

Urodynamic evaluations are employed to obtain quantitative data regarding the bladder. In general urodynamic testing depicts the relationship of bladder pressure to volume of contained fluid, bladder capacity, bladder compliancy (the ability of the bladder to accommodate increasing volumes), bladder pressure during urination (pressure/flow study) and times under different conditions.

### SUMMARY OF THE INVENTION

The present invention overcomes the deficiencies of the background by providing a device, system and method for intravesical urodynamic analysis.

Embodiment of the present invention provides a device for placement and/or deployed within the lumen of the urinary bladder of a patient the device configured to determine urodynamic parameters utilized to perform a urodynamic analysis, while the device is configured to float independently and/or be buoyant within the urinary bladder. The device is characterized in that it comprises a sensor module configured to preform volume measurements and determine the liquid volume of the urinary bladder within which the device is placed.

Optionally the device may be configured as a unitary device having a single housing, deployed within the lumen of the urinary bladder.

Optionally the device may be configured as a split housing device having at least two housing comprising: a first housing defining an internal portion disposed within the lumen of the urinary bladder and a second housing, defining an external portion disposed external to the urinary bladder.

Optionally and preferably the sensor module comprises at least one and more preferably a plurality of sensors including at least one ultrasound sensors, provided for determining the bladder volume which may be utilized to infer at least one urodynamic parameters for example including but not limited to urinary liquid bladder volume, urine flow, bladder pressure, the like or any combination thereof.

Optionally the urinary bladder volume may be determined utilizing a plurality of sensors for example including but not limited to at least one or more sensor including but not limited to ultrasound sensor, optical sensor, a Helmholtz resonance sensor, piezoelectric sensor, Radio Frequency (‘RF’), Infrared (‘IR’) (sensor, the like or any combination thereof.

Optionally the urinary bladder liquid volume may be determined utilizing Helmholtz resonance equations, wherein the resonance frequency and volume of the urinary bladder may be measured and/or determined so as to infer the urinary bladder's liquid volume.

Optionally and preferably a plurality of ultrasound sensors may be provided for scanning at least a portion of the urinary bladder to provide an internal image of the urinary bladder. Optionally the ultrasound sensors are provided for scanning at least a portion of the urinary bladder to determine its internal volume without providing an image.

Optionally a plurality of ultrasound sensors may be provided and dispersed along the device housing so as to facilitate determination the dimensions of the urinary bladder in at least three dimensions for example including X, Y and Z axis. Most preferably the ultrasound sensor provides for estimating the internal volume of the urinary bladder. Optionally and preferably the internal volume of the urinary bladder may then be correlated to at least one or more urodynamic parameter for example including but not limited to urinary flow, liquid volume, bladder pressure, the like or nay combination thereof.

The sensor module includes a pressure sensor for measuring the bladder pressure. Optionally the sensor module may further comprise optional sensors for example including but not limited to flow sensor, flow-meter, temperature sensor, optical sensor, heart rate sensor, pH-meter, glucose meter, oximeter, accelerometer, gyro sensor, the like or any combination thereof.

Preferably the device may further comprise electronic circuitry comprising at least one or more selected from the group consisting of: communication module, memory module, controller module, and real time clock, the like or any combination thereof.

Optionally the device provides for measuring the urodynamic parameters for example including but not limited to urine flow, internal bladder volume, bladder liquid volume, urinary flow rate, and bladder pressure, the like or any combination thereof.

Optionally and preferably the device provides for measuring the urodynamic parameters in a non-clinical setting and/or environment most preferably a user's natural environment allowing for natural filling over a period of time. Optionally the period of time may be from about 1 hour and up to about 48 hours, more preferably the device may be in use for about 24 hours.

Preferably the device may be introduced into the urinary bladder with an introducing catheter. Optionally the introducing catheter may be a dedicated device provided to associate with the urodynamic device housing and introduced into the urinary bladder.

Although the aforementioned device is described with respect to its uses within the urinary bladder as utilized for urodynamic analysis, the device is not limited to such use and may optionally be configured for placement within any portion of the male or female anatomy capable of receiving it, for example including but not limited to uterus, vagina, fallopian tubes, portion of the gastrointestinal tract, large intestine, esophagus, stomach, anus, nose, mouth, bronchi, respiratory tract, upper respiratory tract, lower respiratory tract, gall bladder, sinuses, any internal cavity, or the like anatomy having a lumen capable of receiving the device.

Embodiments of the present invention provide a system and/or kit for performing urodynamic analysis the system comprising the device according to optional embodiments of the present invention, an introducing catheter, and a processor module. Optionally and preferably the system may further comprise at least one or more abdominal sensors, most preferably provided in the form of at least one or more abdominal pressure sensor and/or transducer. Optionally the abdominal pressure sensor and/or transducer may be provided in the form of a belt. Optionally the abdominal sensor may further comprise surface electrodes optionally for obtaining an electromyogram (‘EMG’) of the abdominal surface. Optionally the abdominal EMG signal may be used to inference and/or correlate with the abdominal pressure.

Optionally the system may further comprise a urine absorption device for determining the amount of urine absorbed therein. Optionally such a urinary absorption device and/or pad (162) that may comprise an urine absorption portion, example in the form of a pad, sponge or the like, that is coupled with sensor capable of determining the volume and/or amount of urine absorbed. Optionally, the sensor may be realized as a weight sensor, volume sensor, optical sensor, wetness sensor, fluid sensor any combination thereof or the like.

Optionally and preferably the processing module may be provided in the form of an external processing unit for example provided in the form of a mobile communication and processing device, smartphone, computer, server, call center, health care provided server, dedicated processing and communication device, mobile telephone, PDA, or the like device preferably comprising display, communication and processing capabilities.

Preferably the processing module provides for communicating with the urodynamic device, optionally utilizing wireless and/or wired communication protocols as is known in the art for example including but not limited to WiFi, Bluetooth, near field, RF, IR, wired, or the like. Optionally and preferably the processing module may provide for communicating both with the urodynamic device and the abdominal pressure sensor and provides for analyzing both to determining the urodynamic parameters and providing the urodynamic analysis.

Optionally the system may further comprise and utilize optional auxiliary devices to facilitate performing urodynamic analysis. Optionally auxiliary device may for example include but is not limited to urinary absorbent pads, external catheters, urinary collection bags, stimulating electrodes, surface electrodes, implantable urinary incontinence devices, incontinence cuff and pump or the like.

An optional embodiment of the present invention provides a method for determining a plurality of urodynamic parameter and performing urodynamic analysis based on data provided from the device according to the present invention, most preferably comprising the internal volume of the urinary bladder. Most preferably the method according to the present invention provides urodynamic analysis in a non-clinical setting allowing for preforming urodynamic analysis by way of natural filling.

Embodiments of the present invention provide a device configured for placement within the urinary bladder for performing intravesical urodynamic measurements, the device comprising: an external housing provided from biocompatible materials; a sensor module including a plurality of ultrasound sensors, and at least one pressure transducer; the sensor module functionally coupled with electronic circuitry; and wherein the electronic circuitry comprises a communication module; memory module, and a controller module;

Optionally the housing may be configured to associate with an introducing catheter.

Optionally the device may be configured to be placed within the urinary bladder with the introducing catheter.

Optionally the sensor module may comprise at least 2 ultrasound sensors. Optionally the sensor module may comprise at least 4 ultrasound sensors. Optionally the sensor module may comprise at least 6 ultrasound sensors.

Optionally and preferably the external housing may be configured to assume a capsule shape. Optionally the capsule shape may be configured to have a length of about 10 to about 18 mm and width of about 3 mm to about 8 mm.

Optionally the device may further comprise at least one internal housing. Optionally the at least one internal housing may be provided for containing a fluid and provided in the form of a fluid filled container. Optionally the internal housing may be disposed centrally within the housing.

Optionally the internal housing may be filled with a flowing fluid for example including but not limited to a liquid, gas, air, gel, mixture, saline, the like or any combination thereof.

Optionally the internal housing comprises a filling port and catheter.

Optionally the filling port and catheter may be utilized to fill the internal housing from an external fluid source.

Optionally the external fluid source may be provided from a syringe.

Optionally the device may further comprise a plurality of internal housing compartments in the form of a fluid filled bladder. Optionally each compartment may comprise an individual filling port and associated catheter. Optionally the plurality of internal housing compartments may have a common filling catheter.

Optionally the sensors of the sensor module may be distributed along the external surface of the external housing.

Optionally the electronic circuitry may be disposed along the external surface of the external housing.

Optionally the electronic circuitry and the sensor module are disposed along the external surface of the external housing.

Optionally and preferably the device is configured to provide measurements of the urinary bladder including at least bladder volume and pressure.

Optionally the external housing may be provided from medical grade silicone.

Optionally the internal housing may be disposed within the external housing.

Optionally the internal housing may be sealed from the external housing.

Optionally the internal housing may be configured to include the sensor module and the electronic circuitry.

Optionally the volume between the external housing and the internal housing forms a bladder that may be filled with a flowing fluid.

Optionally the external housing comprises filling port and catheter provided to fill the bladder with a flowing fluid.

Optionally the filling port and catheter may be utilized to fill the bladder from an external flowing fluid source.

Optionally the external flowing fluid source may be provided in the form of a syringe.

Optionally the device may be configured to be a single use device. Optionally the device may be configured to be a multi-use device.

Optionally and preferably the electronic circuitry comprises a real time clock.

Optionally the sensor module comprises plurality of ultrasound sensors characterized in that they are disposed along the housing so as to enable a scan of each axis including the X, Y, Z axes.

Embodiments of the present invention provide a system for intravesical urodynamic measurement, the system comprising the intravesical urodynamic device, according to an optional embodiment of the present invention, an abdominal sensor belt including at least one pressure sensor, an introducing catheter and an external processing unit in communication with the abdominal pressure sensor and the intra vesical urodynamic device.

Optionally the abdominal sensor may comprise at least one or more EMG electrodes.

Embodiment of the present invention provides a method for obtaining intra vesical urodynamic measurements and parameters from a patient, for a given period of time, the method comprising: Fitting a patient with an intravesical urodynamic device according to optional embodiments of the present invention, optionally and preferably utilizing a delivery catheter;

Fitting a patient with an abdominal sensor including at least one pressure sensor;

Synchronizing measurements between the abdominal pressure sensor and the intra vesical urodynamic device; and

Initiating measurement for the given period of time. Optionally the method may further comprise calibrating the abdominal pressure sensor and the intravesical urodynamic device relative to the urinary bladder. Optionally the calibration may be preceded by emptying the urinary bladder.

Optionally the method may further comprise, initiating communication and synchronization between an external processing unit, the abdominal pressure sensor and the intra vesical urodynamic device; continuous monitoring and communication of urodynamic parameters with the abdominal pressure sensor and the intra vesical urodynamic device; and continuous analysis of the urodynamic parameters with the external processing unit.

Optionally the method may further comprise, recording the measured urodynamic parameters with the abdominal pressure sensor and the intra vesical urodynamic device for a given period of time; removing the abdominal pressure sensor and the intra vesical urodynamic device; communicating the urodynamic parameters to an external processing unit; and analyzing the urodynamic parameters with the external processing unit.

Optionally the given period of time may be about 24 hours.

Embodiments of the present invention provide a method for determining urine flow with the device and/or system according to the present invention, the method comprising determining the urinary bladder volume by scanning the urinary bladder with a plurality of ultrasound transducers and therein measuring the bladder volume and thereafter inferring the liquid volume within the bladder.

Optionally and preferably the urinary bladder is emptied prior to determining the starting urinary bladder volume.

Optionally determining the urinary bladder volume may be performed at a frequency from about 1 Hz up to about 20 Hz. Optionally determining the urinary bladder volume may be performed at a frequency of up to about 20 Hz. Optionally determining the urinary bladder volume may be performed at a frequency from about 5 Hz.

Embodiments of the present invention provide a method for determining bladder pressure with the system according an optional embodiments of the present invention, the method comprising: determining the internal bladder pressure with an internal pressure sensor disposed on the intra vesical urodynamic device; determining the abdominal pressure with the abdominal pressure sensor; and comparing the internal bladder pressure and the external abdominal pressure to determine the urodynamic pressure.

Unless otherwise defined the various embodiment of the present invention may be provided to an end user in a plurality of formats, platforms, and may be outputted to at least one of a computer readable memory, a computer display device, a printout, a computer on a network or a user.

Unless otherwise defined, all technical and scientific terms used herein have the same meaning as commonly understood by one of ordinary skill in the art to which this invention belongs. The materials, methods, and examples provided herein are illustrative only and not intended to be limiting. Implementation of the method and system of the present invention involves performing or completing certain selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1A are schematic graphical representations of urodynamic analysis;

FIG. 1B is a schematic illustrative diagram showing prior art urodynamic measuring system utilized to perform urodynamic analysis;

FIG. 2A is a schematic block diagram of an exemplary device according to an optional embodiment of the present invention;

FIG. 2B is a schematic block diagram of an exemplary device according to an optional embodiment of the present invention;

FIG. 3A-C are schematic illustrations of an exemplary device according to an optional embodiment of the present invention;

FIG. 3D-E are schematic illustrations of an exemplary device depicted in FIG. 3A-C disposed within a urinary bladder, according to an optional embodiment of the present invention;

FIG. 4A is a schematic box diagram illustration of an exemplary system according to an optional embodiment of the present invention;

FIG. 4B is a schematic illustration, showing placement of an exemplary system within the urinary bladder according to an optional embodiment of the present invention;

FIG. 4C is a schematic illustration, showing placement of an exemplary system within the urinary bladder according to an optional embodiment of the present invention;

FIG. 5 is a flowchart depicting a method for determining urodynamic parameters according to an optional embodiment of the present invention; and

FIG. 6 is a flowchart depicting a method for performing urodynamic analysis according to an optional embodiment of the present invention.

### DESCRIPTION OF THE PREFERRED EMBODIMENTS

The principles and operation of the present invention may be better understood with reference to the drawings and the accompanying description.

The following figure reference labels are used throughout the description to refer to similarly functioning components are used throughout the specification hereinbelow.
50 urinary bladder;
52 ultrasound scan lines;
100,101 Urodynamic device;
100c central configuration;
100p peripheral configuration;
101i internal device portion;
101e external device portion;
102 external housing;
104 internal housing/compartment/buoyancy container;
106 pressure sensor;
108 sensor module;
108v volume sensor;
108u ultrasound sensor/transducer;
110 electronic circuitry;
112 controller module;
114 real-time clock;
116 memory module;
118 communication module;
120 filling tube/catheter;
121 filling device/syringe
122 filling port;
150 urodynamic analysis system;
152 abdominal sensor;
154 processing unit/device;
156 Introducing device;
158 Filling device;
160 auxiliary device;

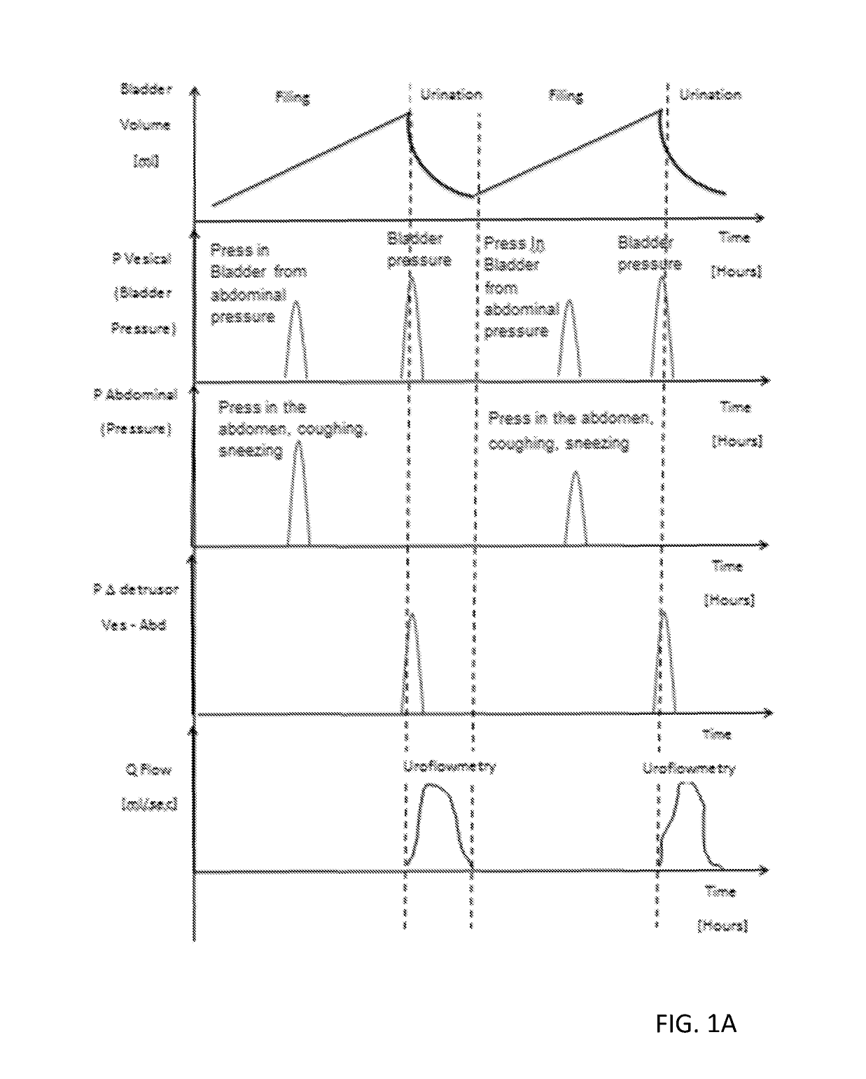

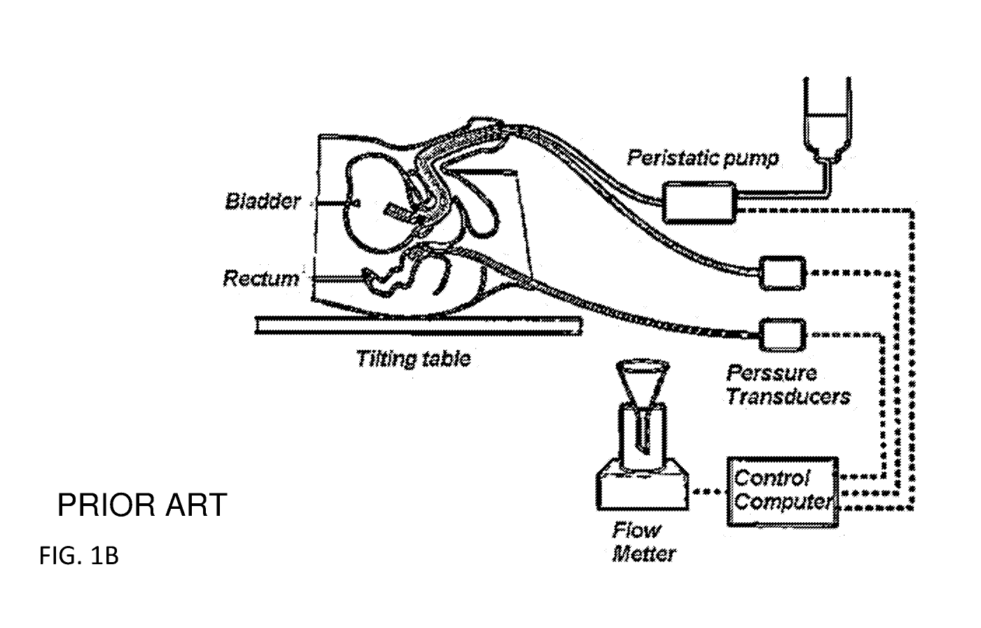

FIG. 1A show a graphical depiction of traditional and/or standard urodynamic analysis results that correlate the relationship between pressure and urine flow over time to determine the type of incontinence, problem at hand, what type of treatment to provide, locate the problem. The upper curve shows bladder volume fluctuation over time both during bladder filling and urination (bladder emptying). The bladder pressure curve shows fluctuation of pressure exerted on the bladder over the same time frame. The abdominal pressure curve shows the change in abdominal pressure over the same time frame. The detrusor pressure curve shows the difference in pressure between the urinary bladder pressure and abdominal pressure that provides an indication of the state of the detrusor muscle. Finally the Qflow curve shows the urinary flow measured in ml/sec (milliliters per second) during urination. The curves depicted in FIG. 1A show a normal bladder activity where the bladder emptying is controlled and not affected by a sudden increased in abdominal pressure, for example as may be expected with incontinence.

FIG. 1B shows state of the art system utilized for performing the urodynamic analysis depicted in FIG. 1A. As evident current system are provided in a clinical setting using a number of invasive measures including a peristaltic pump utilized to fill the bladder while a rectal and urinary bladder pressure transducer are inserted to determine the effect on increasing bladder pressure has on urine flow. As this is a clinical situation it cannot account for all situation that could lead to urinary incontinence for example sudden abdominal pressure increase.

The device system and method of the present invention provide for determining the urodynamic parameters utilized to perform such traditional urodynamic analysis however in a non-clinical setting and/or environment for example an intravesical and/or natural setting environments.

The device, system and method of the present invention is unique in the method by which the urodynamic parameters are determined allowing for seamless determining of the urodynamic parameters in a non-clinical setting such that it is provided in a more comfortable and user friendly environment without the limitations offered by testing under clinical setting.

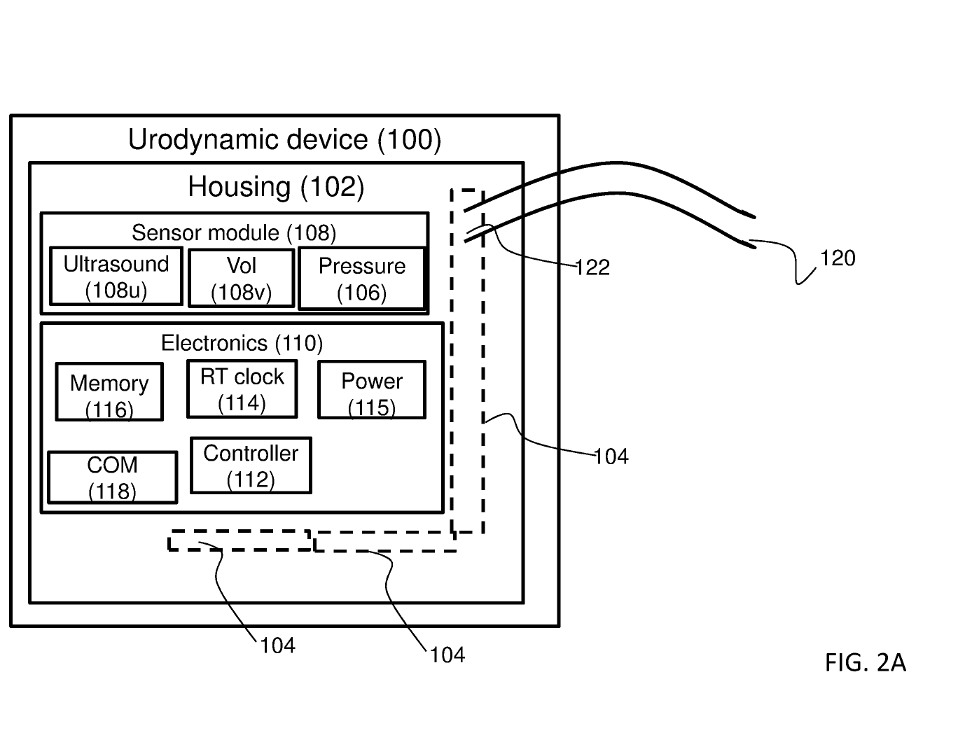

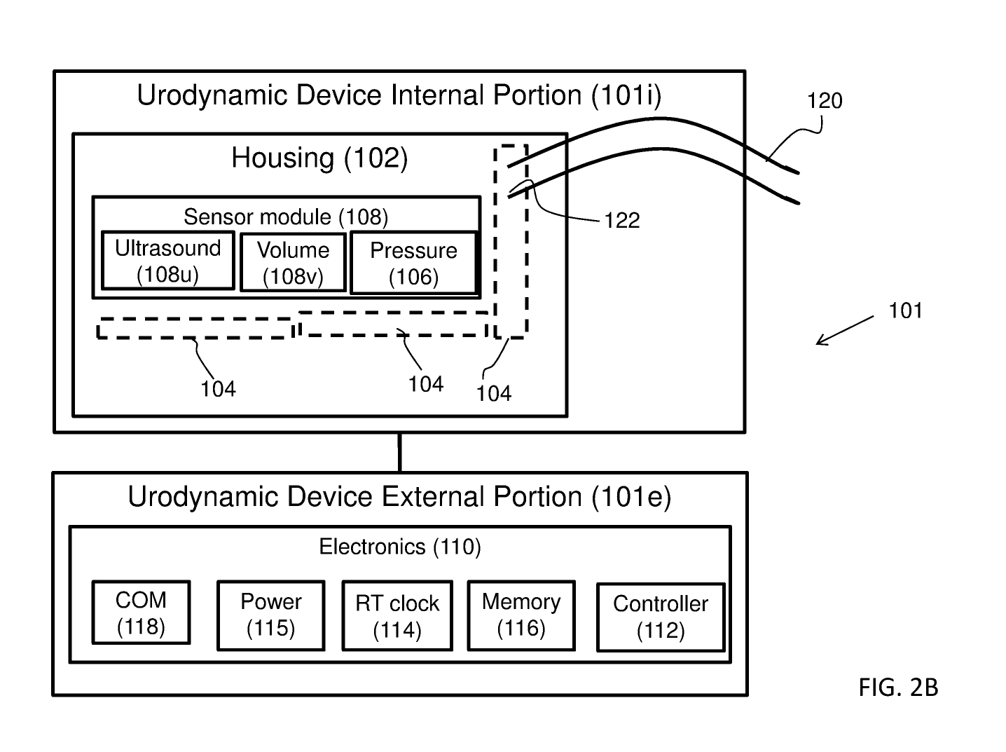

FIG. 2A-B show optional embodiments for a urodynamic device 100,101 according to the present invention, that is characterized in that the device including a sensor modules 108 comprising at least one or more volume sensors 108v that are configured to be deployed internally within the lumen of the urinary bladder for facilitating undertaking a urodynamic analysis under normal physiological conditions, non-laboratory conditions, where the bladder is allowed to fill and empty in a natural surroundings. Optionally and preferably the sensor module 108 provided within a housing intended to be buoyant and/or float within the urinary bladder. Optionally and preferably the housing may be filled with a flowing fluid to provide it with buoyancy within the urinary bladder.

FIG. 2A shows an optional embodiment of device 100 provided in the form of a unitary device that is configured to be completely placed within the lumen of the urinary bladder 50.

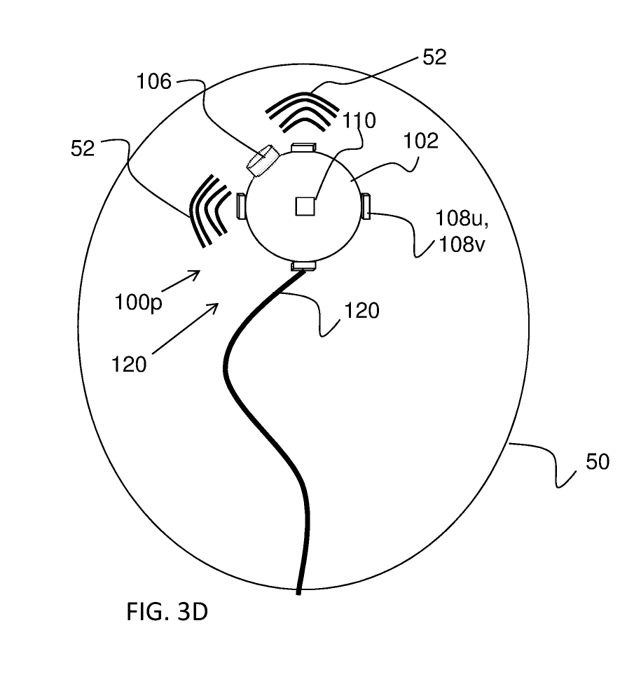

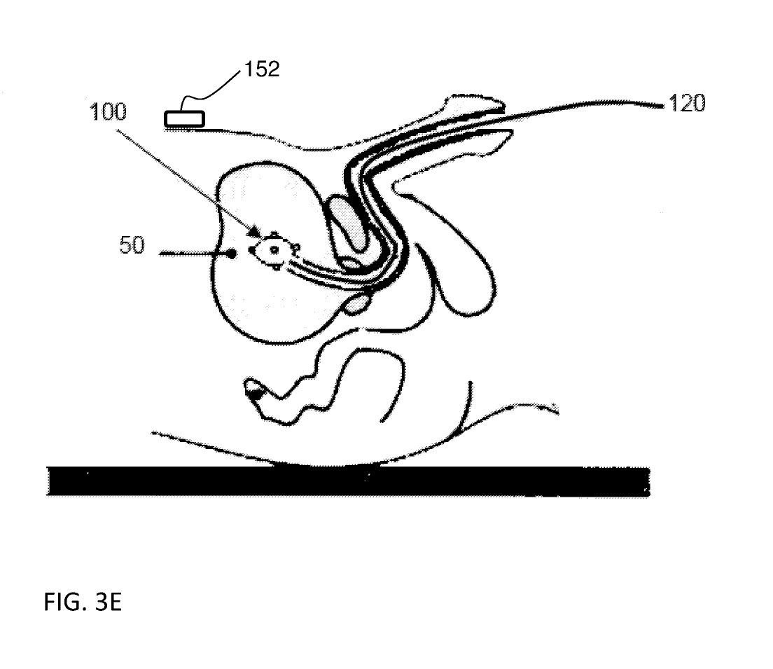

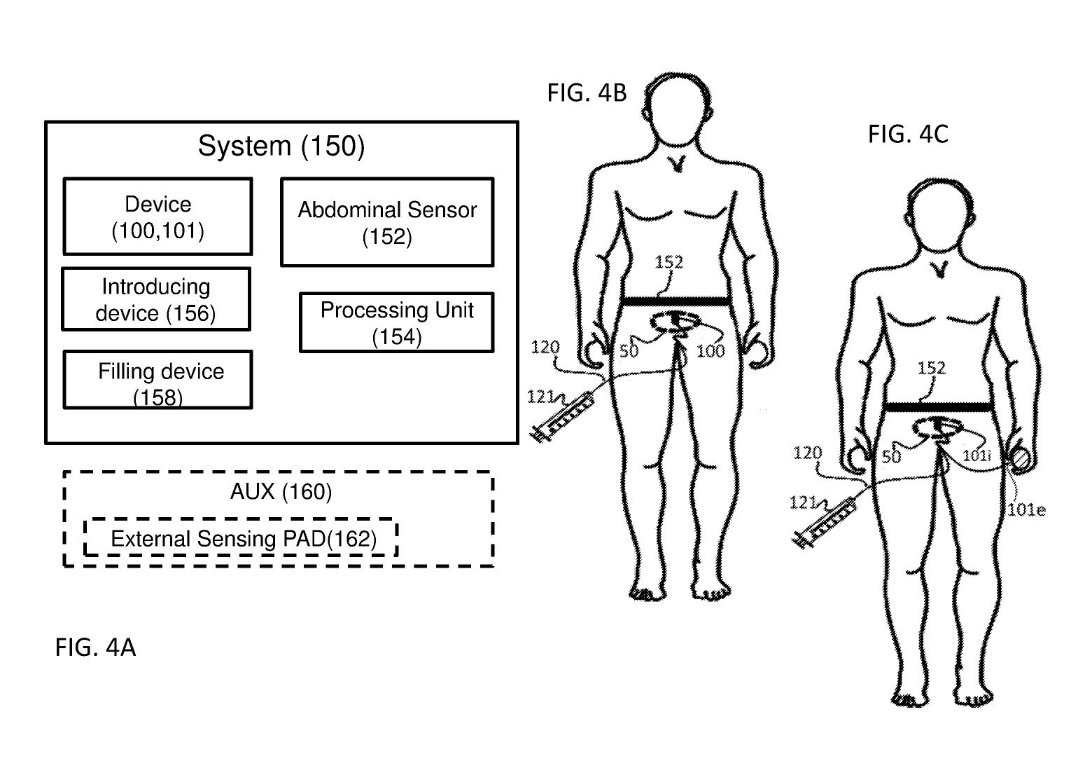

FIG. 2B shows an optional embodiment of device 101 that comprises the same functional modules as device 100 however they are provided in a split housing device 101, having an internal device portion 101i, including sensor module 108, provided for deployment within the lumen of the urinary bladder that is functionally coupled to an external device portion 101e configured to be placed external to the urinary bladder and/or to be placed external to the patient's body. Optionally device 101 along tis external portion 101e is provided with electronic circuitry configured to control and communicate with the internal portion 101i. The split housing device 101 is further shown in FIG. 4C. Optionally external portion 101e may be provided with a human interface for controlling internal portion 101i.

FIG. 2A provides a schematic block diagram of urodynamic device 100 according to an optional embodiment of the present invention. Preferably device 100 is provided to be placed within the urinary bladder 50 (not shown) to facilitate obtaining and determining at least one or more urodynamic parameters that in turn facilitate performing urodynamic analysis. Most preferably device 100 provides for obtaining the urodynamic parameters in a non-clinical setting and/or in an intravesical setting.

Most preferably device 100 is provided such that it may be deployed within the lumen of urinary bladder 50 with an introducing device 156 for example in the form of a catheter. Most preferably the size of device 100 is sufficiently small to allow deployment within bladder 50 for a period of time sufficient to allow for urodynamic analysis under non-laboratory conditions. Optionally the size of device 100 may be configured to have size dimensions of length and width equivalent to about 10 mm (millimeters) up to about 18 mm in length and from about 3 mm to about 8 mm in width.

Preferably device 100 may be introduced by way of minimally invasive procedure utilizing an introducing catheter.

Optionally and preferably device 100 may assume a minimal profile (small) configuration prior to its introduction within the lumen of the urinary bladder, and once deployed within the urinary bladder lumen it is allowed to assume a maximal profile (full size) configuration. Optionally the change form a minimal profile to a maximal profile may be facilitated by way of exposing the device 100 to a triggering signal for example including but not limited to a temperature increase, exposure to a triggering agent such as an electromagnetic signal, a triggering fluid, mechanical trigger, mechanical release, introducing a flowing fluid under pressure such as air under pressure, the like or any combination thereof.

Optionally the change form a minimal profile to a maximal profile is facilitated by way of filling the device housing with a flowing fluid. Optionally and preferably the flowing fluid further provides for rendering device 100 buoyant within the urinary bladder lumen.

Optionally the housing of device 100 may be provided from biocompatible memory shape materials capable of assume at least two profiles for example including but not limited to polymers and/or alloys, nitinol, the like or any combination thereof.

Device 100 includes a sensor module 108, and electronic circuitry 110 that are provided within a housing 102.

Optionally housing 102 may comprise at least one or more internal compartment 104 for receiving and/or containing a flowing fluid to facilitate maintaining buoyancy of device 100 once deployed within the lumen of the urinary bladder 50. Optionally the housing 102 may be provided in the form of a fluid filled container and/or sac.

Optionally housing 102 may be provided as a single external housing.

Optionally housing 102 may further comprise an internal housing 104. Optionally internal housing 104 may be provided from a plurality of sub-compartments arranged within external housing 102.

Most preferably at least one of external housing 102 or internal housing 104 may be utilized to house sensor module 108 and/or electronic circuitry 110

Optionally one of internal housing 104 or external housing 102 may be provided in the form of a container and/or sac that is provided for storing a flowing fluid. Optionally the housing 102 or 104 may be provided in the form of fluid filled container that is sealed from the other housing.

Optionally housing 102 may be provided from flexible, balloon like and/or pliable materials for example including but not limited to in the form of a pliable balloon for example in the form of an angioplasty balloon.

Preferably the buoyancy container 104 may be filled utilizing a filling catheter 120 and a filling port 122. Preferably filling catheter 120 and port 122 are provided so as to allow the filling of the fluid filled container with a flowing fluid, with an optional filling device 158 (FIG. 4A), for example in the form of a syringe 121 (FIG. 4B, 3E). Optionally the fluid filled container may be filled with any fluid for example including but not limited to a liquid, water, gel, gas, saline, solution, or the like flowing fluid preferably configured to provide device 100 with the required buoyancy within the urinary bladder. Preferably the flowing fluid provides device 100 with the required buoyance within the lumen of the urinary bladder such that it is always buoyant within the aqueous environment within the urinary bladder.

Optionally device 100 may be configured to have a central configuration 100c wherein electronic circuitry 110 and sensor module 108 may be disposed centrally within device 100 while the periphery is provided with a flowing fluid buoyancy fluid. Optionally in such a central configuration electronic circuitry 110 and sensor module 108 may be disposed within an internal housing 104 that is sealed from the fluid filled external housing 102.

Optionally device 100 may be configured to have a peripheral configuration 100p wherein electronic circuitry 110 and sensor module 108 may be disposed along the external housing 102 surface defining the periphery of device 100, while the center of the device is provided with a flowing fluid buoyancy fluid within an internal container 104.

Optionally device 100 may be configured to have a mixed configuration where each of electronic circuitry module 110 and sensor module 108 are individually sealed while at least portions thereof may be functionally associated and/or coupled with one another.

Most preferably electronic circuitry 110 comprises a plurality of electronic modules rendering device 100 operation. Electronic circuitry 110 preferably comprises at least one or more functional units selected from the group for example including but not limited to controller module 112, real-time (‘RT’) clock, memory module 116 and communication module (COM) 118. Most preferably controller module 112, comprising a processor, and power source module 115, are provided for controlling and powering device 100. Preferably controller module 112 provides for controlling the overall function of device 100 and coordinates functionality between electronic circuitry 110 and sensor module 108.

Optionally power module 115 may be provided in optional form for example including but not limited to a battery, induction coil, the like or any combination thereof. Optionally power module 115 may be re-energized and/or recharged before and/or after device 100 has been removed from the urinary bladder. Optionally power module 115 may be configured to be recharged during deployment while disposed within the urinary bladder for example by way of remote and/or wireless and/or contactless electromagnetic energy source for example including but not limited to electromagnetic induction, magnetic induction, RF (radio frequency) signal, NFC (near field communication) signals, the like or any combination thereof as is known in the art. Optionally power

Optionally communication module 118 provides for communicating with external device preferably according to wireless and/or contactless technology and/or protocols as is known in the art for example including but not limited to Bluetooth, WiFi, Near Field Communication (NFC), optical communication, acoustic communication, any combination thereof or the like.

Optionally memory module 116 provides optional forms of memory for device 100 to allow all data and communication to be stored. Optionally memory module 116 may be provided in optional forms as is known in the art for example including but not limited to flash memory, volatile memory, non-volatile memory, the like or any combination thereof. Optionally memory module 116 is provided to continuously store data relating to device 100 as soon as it is deployed within the lumen of the urinary bladder. Optionally data stored by module 116 may optionally be communicated and/or transferred and/or downloaded to an external device utilizing communication module 118. Optionally data stored by module 116 may optionally be communicated and/or transferred and/or downloaded to an auxiliary and/or external device by means of wired and/or wireless association and/or coupling. Optionally memory module 116 may be configured to store and/or gather data once deployed within the urinary bladder lumen and download and/or communicated the stored data once removed from the bladder 50.

Electronic circuitry 110 preferably comprises a real time clock (RT) 114 to facilitate accurate timing measurement of device 100.

Device 100 most preferably includes a sensor module 108 that includes at least one or more sensors provided for determining the urinary bladder volume and/or the liquid volume within the urinary bladder. Sensor module preferably comprises at least one volume sensor 108v that is configured to determine at least one of the urinary bladder volume or the liquid volume within the urinary bladder. Optionally volume sensor 108v may be provided to determine the volume in any manner. Optionally volume sensor 108v may be realized in the form of a battery of ultrasound sensors 108u and/or a Helmholtz resonance sensor and/or a magnetic impedance sensor, the like or any combination thereof.

Preferably sensor module 108 includes at least one and more preferably a plurality of ultrasound transducers 108u utilized to determine the volume of the urinary bladder and/or the liquid volume within the urinary bladder.

Optionally sensor module 108 may further comprise a pressure sensor 106, preferably provided to determine the internal bladder pressure of bladder 50.

Optionally sensor module 108 may further comprise optional sensors for example including but not limited to flow sensor, flow-meter, temperature sensor, optical sensor, heart rate sensor, pulse-oximeter, accelerometer, gyro sensor, the like or any combination thereof

Ultrasound transducers 108u most preferably provided for determining the internal volume of urinary bladder 50. Most preferably ultrasound transducers 108u are in functional association with at least a portion of electronic circuitry 110 to render them functional.

Optionally ultrasound transducers 108u may be provided in independent form wherein they are rendered functional without being functionally associated with electronic circuitry 110.

Optionally ultrasound transducers 108u may be positioned along the external surface of housing 102, preferably to provide a three dimensional depiction of the urinary bladder 50. Most preferably a plurality of ultrasound transducers 108u may be disposed along device 100 to provide a three dimensional depiction and/or image of bladder 50. Optionally at least one transducer 108u is provided for scanning and/or directionally scanning along each of the three dimensional axis X, Y, Z. Optionally and more preferably at least two transducers 108u are provided for scanning and/or directionally scanning along each of the three dimensional axis X, Y, Z, therein utilizing a at least 6 transducers

Preferably ultrasound transducers 108u provide an internal image of the size, shape of the bladder in non-clinical setting and/or natural filling conditions, providing an indication of the liquid volume of the bladder.

Optionally ultrasound transducers 108u may be controlled via controller module 112. Optionally the frequency and timing of activating ultrasound transducers 108u may be controlled via controller module 112. Optionally transducers 108u may be activated in any manner for example including but not limited to sequentially, simultaneously, groups, the like or any combination thereof. Optionally transducers 108u may be controlled by an external device 154 (FIG. 4A) via communication with electronic circuitry 110, wherein device 154 may be controlled by a user and/or a computer.

FIG. 2B shows split housing device 101 having an internal portion 101i and an external portion 101e. Preferably internal portion 101i comprises sensor module 108 including at least one or move volume sensor 108v and is configured to be placed within the lumen of the urinary bladder. Preferably external portion 101e comprises electronic circuitry 110 that is functionally associated with internal portion 101i such that external portion 101e is configured to control and/or power internal portion 101i and therein functioning in the same manner as electronic module 110 described with FIG. 2A.

Optionally external portion 101e and internal portion 101i may be functional coupled with one another utilizing wired leads.

Optionally external portion 101e and internal portion 101i may be functional coupled with one another utilizing wireless and/or contract-less communication and/or data transfer protocols as is known in the art for example including but not limited to NFC, Bluetooth, the like or any combination thereof.

Preferably internal portion 101i comprises an external housing 102 and at least one or more internal housings 104 that may be filled with a flowing fluid for example with a catheter 120, for example as previously described.

Preferably internal portion 101i may be introduced by way of minimally invasive procedure utilizing an introducing catheter.

Optionally and preferably internal portion 101i may assume a minimal profile (small) configuration prior to its introduction within the lumen of the urinary bladder, and once deployed within the urinary bladder lumen it is allowed to assume a maximal profile (full size) configuration. Optionally the change form a minimal profile to a maximal profile may be facilitated by way of exposing the internal portion 101i to a triggering signal for example including but not limited to a temperature increase, exposure to a triggering agent such as an electromagnetic signal, a triggering fluid, mechanical trigger, mechanical release, introducing a flowing fluid under pressure such as air under pressure, the like or any combination thereof.

Optionally the change form a minimal profile to a maximal profile is facilitated by way of filling the device housing with a flowing fluid. Optionally and preferably the flowing fluid further provides for rendering internal portion 101i buoyant within the urinary bladder lumen.

Optionally the housing of internal portion 101i may be provided from biocompatible memory shape materials capable of assume at least two profiles for example including but not limited to polymers and/or alloys, nitinol, the like or any combination thereof.

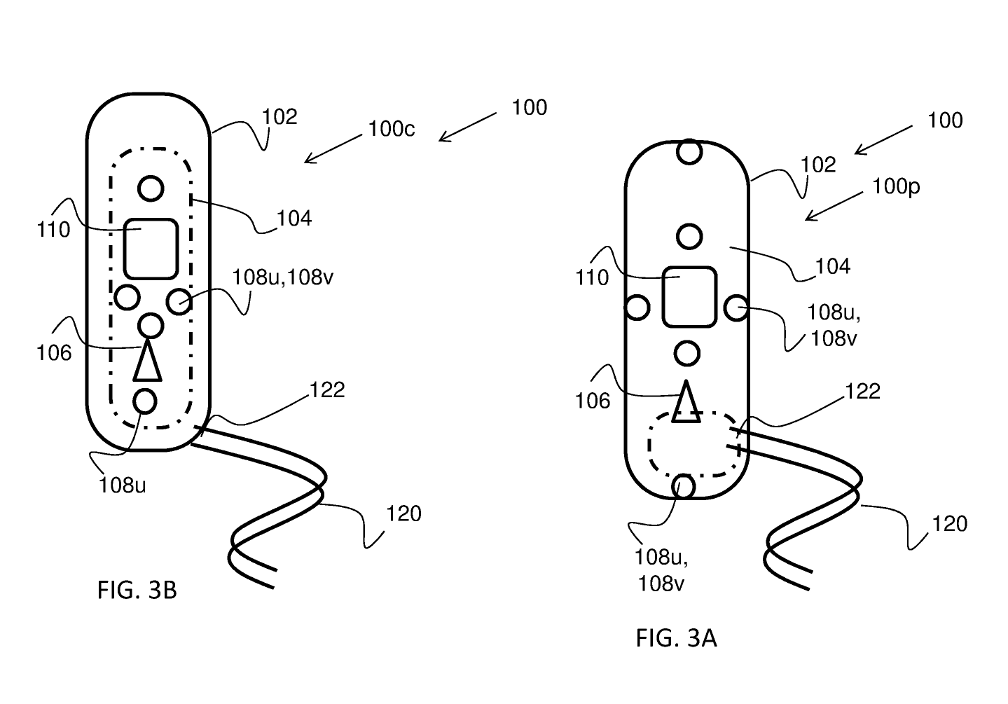

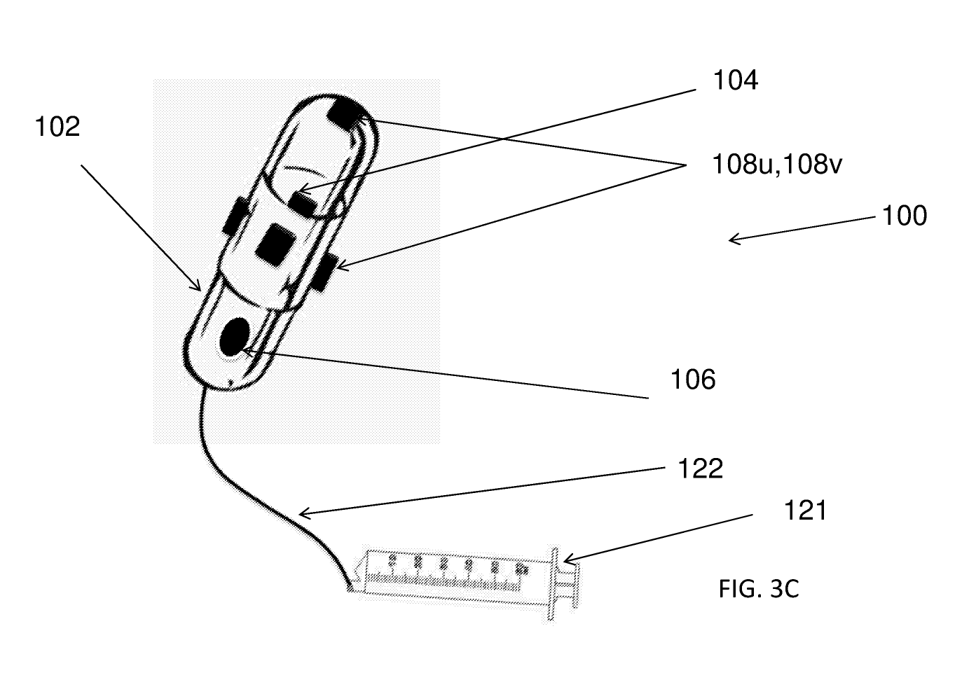

FIG. 3A shows an optional schematic illustrative depiction of device 100, described in FIG. 2A, in a peripheral configuration 100p, where external housing 102 is provided in an optional form of a capsule, for example as shown. FIG. 3A shows a plurality of ultrasound transducers 108u positioned along the external surface of housing 102, therein providing an optional peripheral configuration 100p.

Optional buoyancy container 104 may be disposed within external housing 102 and filled with a buoyancy flowing fluid via port 122 and filling tube 120. Optionally as container 104 is filled it expands while sealed from housing 102.

FIG. 3B shows an optional schematic illustrative depiction of device 100, described in FIG. 2A, in a central configuration 100c, where electronic circuitry 110 and members of sensor module 108 are disposed centrally within an internal housing 104. Optionally in the central configuration 100c the peripheral space defined between housing 104 and external housing 102 may be filled with a flowing fluid, preferably to provide device 100 with buoyancy. Optionally the peripheral space is filled via a filling port 122 and through a filling catheter 120 attached to the external surface of external housing 102, for example as shown.

FIG. 3C shows an optional schematic illustrative diagram of device 100 with a filling device 121 shown in the form of a syringe. Optionally filling device may be provided in optional forms for example including but not limited to a pump, syringe, or the like.

FIG. 3D shows a cross sectional view of device 100 in the peripheral configuration 100p, deployed within the lumen of the urinary bladder 50. As previously described ultrasound transducers 108u are disposed along the external surface of housing 102. Most preferably each transducer 108u produces an ultrasound scan beam 52 to enable estimation of the internal volume of bladder 50. Most preferably transducers 108u provide a scan 52 in each of the three dimensional axis X, Y, Z to facilitate determination and/or estimate of the internal volume of bladder 50. Optionally the frequency and timing of each ultrasound transducer 108u may be controlled with controller module 112 disposed in electronic circuitry 110. Optionally ultrasound processing may be provided with controller module 112, in an online and/or substantially real time manner, while device 100 is deployed within the lumen of bladder 50.

Optionally ultrasound processing may be provided by an external processor and device, for example processing unit 154 described in FIG. 4A and/or an auxiliary unit 160 described in FIG. 4A.

Optionally data relating to the ultrasound transducer 108u may be recorded and/or stored with memory module 116. Optionally the recorded data may be processed to facilitate determining the urodynamic parameters and perform urodynamic analysis after device 100 has been removed and/or recovered from bladder 50.

Optionally and preferably pressure sensor 106 provides for determining the bladder pressure.

FIG. 3D further shows filling tube and/or catheter 120 that facilitates maintaining buoyancy of device 100 within bladder 50, by introducing an optional buoyancy flowing fluid through catheter 120. Optionally buoyancy flowing fluid may be delivered with a syringe or the like optional filling device 158. Most preferably buoyancy flowing fluid provides for maintaining device 100 buoyant while deployed within the lumen of bladder 50.

FIG. 3E shows an optional device 100 disposed within a urinary bladder 50. Device 100 may be utilized in conjunction with an abdominal sensor 152 forming an optional urodynamic analyzing system 150, as described in more detail in FIG. 4A-B.

FIG. 4A shows a block diagram of system 150 according to the present invention providing urodynamic analysis and urodynamic parameter determination. System 150 includes urodynamic device 100, an introducing device 156 and processing unit 154.

Most preferably introducing device 156 provides for introducing device 100 into the urinary bladder. Optionally device 156 may be provided in optional forms for example including but not limited to a catheter, a dedicated device or the like device capable of non-invasively introducing device 100 into the urinary bladder 50. Optionally external housing 102 may be configured to interface with at least a portion of introducing device 156 so as to allow introducing device 156 to carry device 100 into bladder 50 that is optionally lead through the urethra.

Most preferably processing unit 154 may be provided in the form of a computer or the like device comprising display, communication and processing capabilities, that may be in communication with device 100 for example via communication module 118. Optionally processing unit 154 may for example be provided in optional forms for example including but not limited to mobile communication and processing device, smartphone, computer, server, call center, health care provided server, dedicated processing and communication device, mobile telephone, PDA, or the like device preferably comprising display, communication and processing capabilities.

Optionally and preferably system 150 further includes an abdominal sensor 152 preferably provided in the form of a pressure sensor utilized to determine the abdominal pressure during measurement with device 100. Preferably such an abdominal pressure sensor provides for determining and/or comparing the pressure exerted by the user on the bladder during optional incontinence events, for example laughing, coughing, running or the like. Preferably abdominal sensor comprises at least two or more topical abdominal pressure sensors.

Optionally abdominal sensor 152 may be provided in a belt and/or belt-like form. Optionally abdominal sensor 152 may further comprise EMG surface electrodes.

Preferably abdominal sensor 152 may be in communication with at least one of device 100 and/or processing unit 154. Most preferably both abdominal sensor 152 and device 100 may be in communication with processing unit 154. Optionally and preferably processing unit 154 may provide for synchronization between abdominal sensor and device 100.

Optionally and preferably system 150 may further comprise a filling device 158. Device 158 provides for filling and/or controlling the level of flowing fluid within device 100, for example within buoyancy container. Most preferably the level of the flowing fluid may be controlled so as to allow device 100 to be buoyant while deployed within bladder 50. Preferably device 158 may be directly and/or indirectly associated with filling catheter 120 and

System 150 may optionally further comprise an optional auxiliary device 160. Optionally auxiliary device 160 may facilitate performing urodynamic analysis. Optionally auxiliary device 160 may be provided in various forms for example including but not limited to urinary absorbent pads (162), external catheters, urinary collection bags, stimulating electrodes, surface electrodes, implantable urinary incontinence devices, incontinence cuff and pump or the like or any combination thereof.

An optional absorbent pad 162 may be utilized to facilitate urodynamic analysis. Optionally the absorbent pad may be provided in the form of singe use underwear, feminine napkins, feminine sanitary pad, the like or any combination thereof.

Optionally a urine absorption device and/or pad 162 may be utilized to absorb and/or collect urine that leaks while utilizing device 100. Optionally and preferably device and/or pad 162 is fit with sensors capable of determining the amount of urine absorbed thereon. Optionally the urinary absorption device and/or pad 162 may comprise a urine absorption portion, example in the form of a pad, sponge or the like, that is coupled with sensor capable of determining the volume and/or amount of urine absorbed. Optionally, the sensor may be realized as a weight sensor, volume sensor, optical sensor, wetness sensor, fluid sensor any combination thereof or the like.

FIG. 4B shows an illustrative diagram of placement of system 150 described in FIG. 4A utilizing unitary device 100 depicted in FIG. 2A, the system comprising device 100 that is placed within a urinary bladder 50 and an external abdominal sensor 152 utilized to render a urodynamic analysis. Device 100 is preferably non-invasively placed within the urinary bladder 50 with an introducing device 156 (not shown) and is thereafter optionally and preferably rendered functional with a filling device 158, shown in the form of a syringe 121. Preferably device 100 is configured to be buoyant within the lumen of the urinary bladder 50.

FIG. 4C shows an illustrative diagram of placement of system 150 described in FIG. 4A utilizing split housing device 101 depicted in FIG. 2B, the system comprising split housing device 101 including internal portion 101i and external portion 101e, as shown. Internal portion 101i is placed and/or deployed within the internal lumen of the urinary bladder 50, preferably rendering it buoyant therein; and an external abdominal sensor 152 are collectively used to render/perform a urodynamic analysis. Internal portion 101i is preferably non-invasively placed within the urinary bladder 50 with an introducing device 156 (not shown) and is thereafter optionally and preferably rendered functional with a filling device 158, shown in the form of a syringe 121.

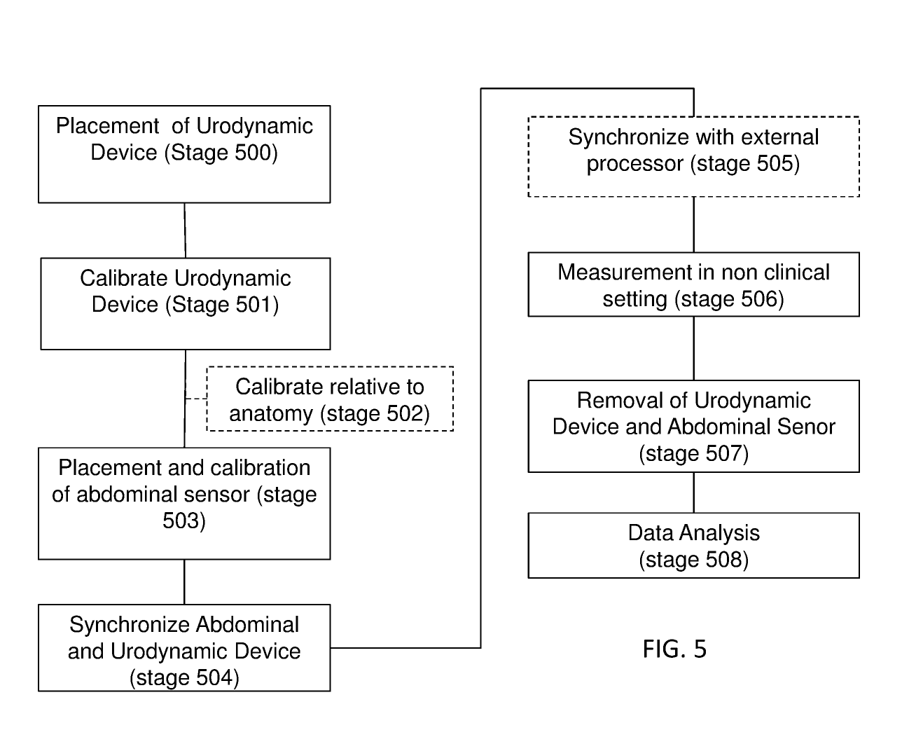

FIG. 5 shows a flowchart depicting an optional method for performing urodynamic analysis utilizing device 100 within system 150, previously described. First in stage 500, urodynamic device 100 is deployed and/or placed within urinary bladder 50 preferably utilizing an introducing device 156, for example in the form of an introducing catheter or the like dedicated device.

Next in stage 501, urodynamic device, now deployed within bladder 50, is calibrated. Optionally during calibration a starting measurement of the internal volume of bladder 50 is determined. Optionally following deployment device 100 within bladder 50, a user may be requested to empty bladder 50.

Next in an optional stage 502 device 100 may be further calibrated relative to bladder anatomy 50 so as to ensure that starting measurements are as accurate as possible.

Next in stage 503 abdominal sensors 152, most preferably including at least one or more abdominal pressure sensors, is associated with a user. Optionally abdominal sensors 152 may be provided in a belt form comprising at least two or more abdominal sensors. Optionally abdominal sensor 152 may further comprise EMG electrodes.

Next in stage 504 both device 100 and abdominal sensor 152 are simultaneously calibrated to ensure that measurements provided with device 100 and abdominal sensor 152 are substantially simultaneously recorded therein allowing correlation and most preferably time synchronization between internal bladder pressure and urodynamic parameters. Most preferably synchronization is further provided relative to at least one processor selected from external unit 154 and/or electronic circuitry 110 control module.

Next in an optional stage 505 a further synchronization is provided between device 100 and external unit 154.

Next is stage 506 device 100 once deployed within the lumen of the urinary bladder is utilized start measuring optionally for a given length of time Optionally and most preferably measurement is provided in a non-clinical and/or intravesical setting.

Next in stage 507, following urodynamic measurement, device 100 may be safely removed. Next in stage 508, following removal of device 100 analysis is performed on the accumulated data. Optionally data from device 100 is may communicated and/or download so as to allow for urodynamic data processor and analysis.

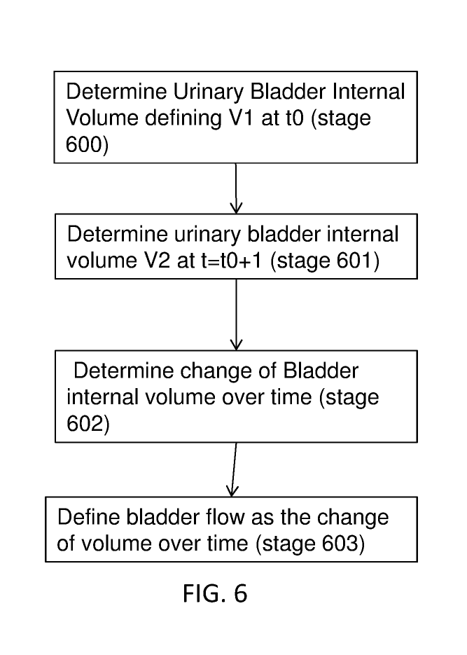

FIG. 6 shows a flowchart according to an optional embodiment of the present invention provided for determining urodynamic parameters from the bladder's internal volume of measurement facilitated urodynamic device 100 deployed within bladder 50. Most preferably device 100 facilitates determination of urodynamic parameters required for providing urodynamic analysis, for example including but not limited to bladder pressure, liquid volume and urine flow. Most preferably these parameters are determined utilizing a plurality of ultrasound transducers 108u to determine internal bladder volume and/or liquid volume. Optionally the internal bladder pressure data may be derived by analysis of the bladder volume data and/or optionally and preferably may be provided from pressure sensor 106, as previously described.

First in stage 600 an initial internal volume V1 at a time t0 of bladder 50 is determined by ultrasound scanning of the bladder utilizing a plurality of ultrasound transducers 108u to provide a three dimensional depiction of bladder within which device 100 is deployed. Optionally V1 may be determined under controllable conditions where bladder 50 is empty, provided by measuring following emptying of the bladder under controlled conditions.

Next in stage 601, at a controllable time interval ultrasound transducers 108u are employed to determine a second internal bladder volume V2 at time t1. Optionally the scanning frequency and timing ultrasound transducers 108u may be controlled by controller module 112.

Most preferably stage 601 is repeated over a given period of time equal to the testing time from 1 hour and up to about 48 hours and more preferably up to about 24 hours.

Next in stage 602, determine the change in internal volume V2−V1 over time interval t=t0−t1 is determined to define the urinary flow over the entire testing period, in stage 603. Optionally the change in volume over time may be further utilized to determine the internal bladder pressure.

While the invention has been described with respect to a limited number of embodiment, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not described to limit the invention to the exact construction and operation shown and described and accordingly, all suitable modifications and equivalents may be resorted to, falling within the scope of the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

Section headings are used herein to ease understanding of the specification and should not be construed as necessarily limiting.

While the invention has been described with respect to a limited number of embodiments, it will be appreciated that many variations, modifications and other applications of the invention may be made.

1-76. (canceled)

77. An intravesical urodynamic device for deployment within a urinary bladder and being buoyant within the urinary bladder lumen, the device provided for performing intravesical urodynamic measurements in a non-clinical setting allowing for urodynamic measurements under normal physiological conditions including natural filling and emptying of the urinary bladder, the device comprising:
a) an external housing comprising a biocompatible material, configured to assume a capsule-like shape and to be wholly placed with the lumen of the urinary bladder;
b) an internal housing forming a fillable fluid bladder having a filling port coupleable to a catheter, the internal housing being disposed within the external housing, and being capable of receiving a liquid for filling the bladder with a liquid via the filling port from an external fluid source;
c) electronic circuitry module disposed within the device external housing, the electronic circuitry having at least a communication module; memory module, and a controller module;
d) a sensor module having at least one volume sensor and at least one pressure transducer;
the sensor module electrically coupled with the electronic circuitry module; wherein the at least one volume sensor is selected from a resonance sensor utilized to determine the resonant frequency and volume of the urinary bladder, and/or a plurality of ultrasound sensors disposed along the device external housing so as to enable a scan of each axis including the X, Y, Z axes; and
e) wherein the device is filled with a liquid selected to render the device buoyant within the urinary bladder lumen.

78. The device of claim 77 further comprising a volume sensor selected from the group consisting of: optical sensor, piezoelectric sensor, Radio Frequency (‘RF’), Infrared (‘IR’) sensor, and any combination thereof.

79. The device of claim 77 wherein the external housing is configured to associate with an introducing catheter provided to place the device within the lumen of the urinary bladder.

80. The device of claim 77 wherein the capsule shape is configured to have a length of about 10 to about 18 mm and width of about 3 mm to about 8 mm.

81. The device of claim 77 wherein the internal housing is disposed centrally within the external housing.

82. The device of claim 77 wherein the external fluid source is provided from a syringe.

83. The device of claim 77 further comprising a plurality of internal housing compartments in the form of a fluid filled bladders and wherein each compartment comprises an individual filling port coupleable to a catheter.

84. The device of claim 77 wherein at least one of the sensors of the sensor module is disposed along the external surface of the external housing.

85. The device of claim 77 wherein the internal housing is sealed from the external housing.

86. The device of claim 77 further comprising a power module configured to receive energy from a remote contactless electromagnetic energy.

87. A system for intravesical urodynamic measurement, the system comprising the intravesical urodynamic device according to claim 77, an abdominal sensor belt having at least one pressure sensor, an introducing catheter and an external processing unit in communication with the abdominal pressure sensor and the intravesical urodynamic device.

88. The system of claim 87 wherein the abdominal sensor further comprises at least one EMG (ElectroMyoGraphy) electrode.

89. A method for determining bladder pressure with the system of claim 87, the method comprising:
a) determining the internal bladder pressure with the at least one internal pressure sensor of the intravesical urodynamic device;
b) determining the abdominal pressure with the abdominal pressure sensor;
c) utilizing the internal bladder pressure and the abdominal pressure to determine the urodynamic pressure.

90. A method for obtaining intravesical urodynamic measurements and parameters from a patient, for a given period of time of at least 1 hour, the method comprising,
a) fitting a patient with an intravesical urodynamic device according to claim 77 utilizing a delivery catheter;
b) fitting a patient with an abdominal sensor including at least one pressure sensor;
c) synchronizing between the abdominal pressure sensor and the intravesical urodynamic device; and
d) initiating measurement for the given period of time.

91. The method of claim 90 further comprising calibrating the abdominal pressure sensor and the intravesical urodynamic device relative to the urinary bladder.

92. The method of claim 90 wherein the calibration is preceded by emptying the urinary bladder.

93. The method of claim 90 further comprising:
e) initiating communication and synchronization between an external processing unit, the abdominal pressure sensor and the intravesical urodynamic device;
f) providing continuous monitoring and communication of urodynamic parameters with the abdominal pressure sensor and the intravesical urodynamic device; and
g) providing continuous analysis of the urodynamic parameters with the external processing unit.

94. The method of claim 93 further comprising:
i) recording the measured urodynamic parameters with the abdominal pressure sensor and the intravesical urodynamic device for a given period of time;
ii) removing the abdominal pressure sensor and the intravesical urodynamic device;
iii) communicating the urodynamic parameters to an external processing unit; and
iv) analyzing the urodynamic parameters with the external processing unit.

95. The method of claim 94 wherein the given period of time is about 24 hours.

96. The method of claim 95 wherein determining the urinary bladder volume is performed at a frequency from about 1 Hz up to about 20 Hz.
